(19) United States
(12) Patent Application Publication
(10) Pub. No.: US 2019/0099164 A1
(43) Pub. Date: Apr. 4, 2019

(54) Labeling Method for Medical Samples

(71) Applicant: Green Panther Ventures, Huntington Beach, CA (US)

(72) Inventors: Matt S. Green, Huntington Beach, CA (US); Joseph Daniel Massam, Langhorne, PA (US)

(73) Assignee: Green Panther Ventures, Huntington Beach, CA (US)

(21) Appl. No.: 16/052,435

(22) Filed: Aug. 1, 2018

Related U.S. Application Data
(60) Provisional application No. 62/543,223, filed on Aug. 9, 2017.

(51) Int. Cl. A61B 10/00; B01L 3/00

(52) U.S. Cl. CPC A61B 10/007; B01L 3/545; G01N 2001/007; B65D 2101/0023; B01L 3/50

(57) ABSTRACT

An method for labeling and sealing medical samples prevents tampering, saves time, and reduces user error in matching medical samples to patients. The label has two portions, one portion being detachable and each portion having a barcode containing the patient's information as well as areas for the patient's signature, and/or the label also acts as a tamper strip for the sample container, and/or the patient's information is embedded or pre-printed on the sample container, as well as a label to do the same.

### BACKGROUND

In the practice of modern medicine, there is a constant need to acquire and store samples of matter from the bodies of patients, in order to aid in the process of medical diagnosis and to evaluate the necessity of treatment, further medical tests, or other procedures. These samples must be labeled and sealed in a fashion that prevents tampering and accurately matches the sample with the patient from which it came. Furthermore, the high volume of samples processed and the generally hectic environment of medical facilities create a special need for a method which is quick, simple, and leaves minimal room for user error.

The current process for labeling and sealing medical samples involves many steps and several different labeling elements, such as the sample cup and the patient information strip, that medical personnel must keep track of while storing the sample. First, the collector hands the sample cup and the tamper strip to the patient, with instructions to: 1) initial the tamper strip, 2) sign the back of the strip, and 3) sign the top of the cup. Next, the collector receives the signed tamper strip back from the patient, scans the patient's information into the system from another adhesive strip, and sets the patient's information strip as well as the signed tamper strip aside for later. Lastly, the collector compares the signatures and patient information on the tamper strip and the cup, ensures that the tamper strip, patient information, and cup match, and then seals the sample by placing the tamper strip over the lid of the cup.

Due to the number of steps and elements involved, this method is relatively slow, which detracts from time spent performing another of a multitude of time-critical tasks required of medical personnel. For the same reasons, this process often results in user error, resulting in medical samples being matched to the wrong patient; this has the potential to cause confusion, delays, and even misdiagnosis. Furthermore, these steps can reduce the confidence of the sample provider that the integrity of the sample has been maintained, which can be a source of anxiety for the sample provider. As such, there is a distinct need for a simpler and more streamlined labeling system.

### BRIEF DESCRIPTION OF THE DRAWINGS

FIGS. 1-2 show the double-barcode label and its features.

FIGS. 3-7 illustrate the steps of a sample labeling process using the double-barcode label.

FIGS. 8-9 show an example of the label and tamper-proof seal and its features.

FIG. 10 shows an example of the built-in label and its features.

FIG. 11 illustrates the computer system described herein.

### DETAILED DESCRIPTION OF THE EMBODIMENTS

Double Barcode Label

One way of simplifying the process of storing samples is to reduce the number of elements that the patient must sign, and to reduce the number of elements that the sample collector must keep track of and the number of steps to be performed while carrying out the process. Along these lines, it would also be a benefit to have these steps performed in the sample provider's presence, so that they can witness, and thus have confidence in the chain of custody of the sample. It would also be a benefit for the sample provider to be able to track the chain of custody of the sample. To this end, the label is designed to consolidate the functionalities of multiple different elements into one.

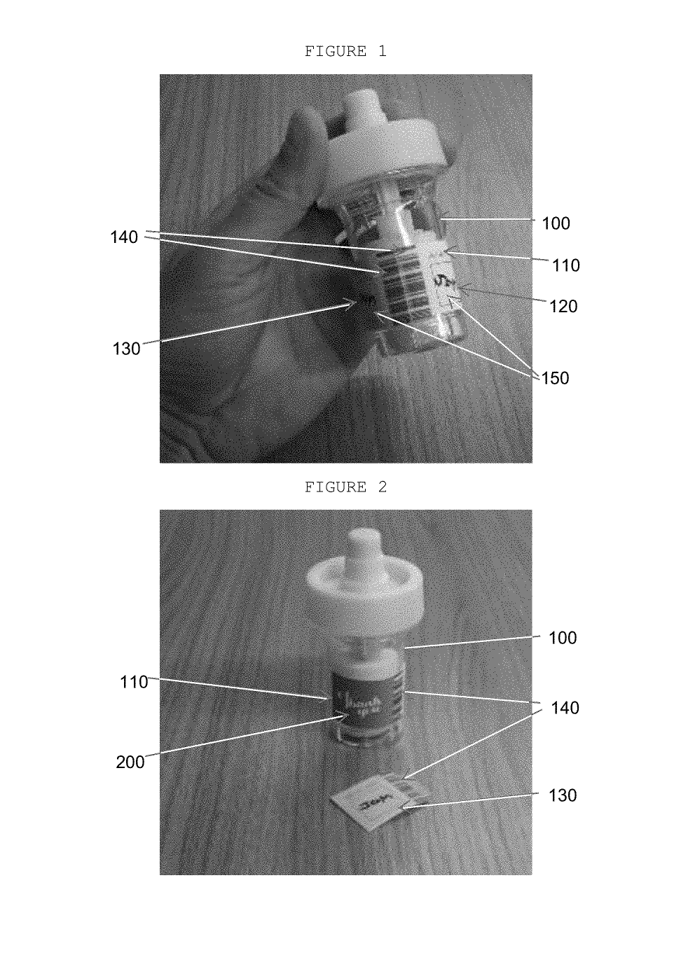

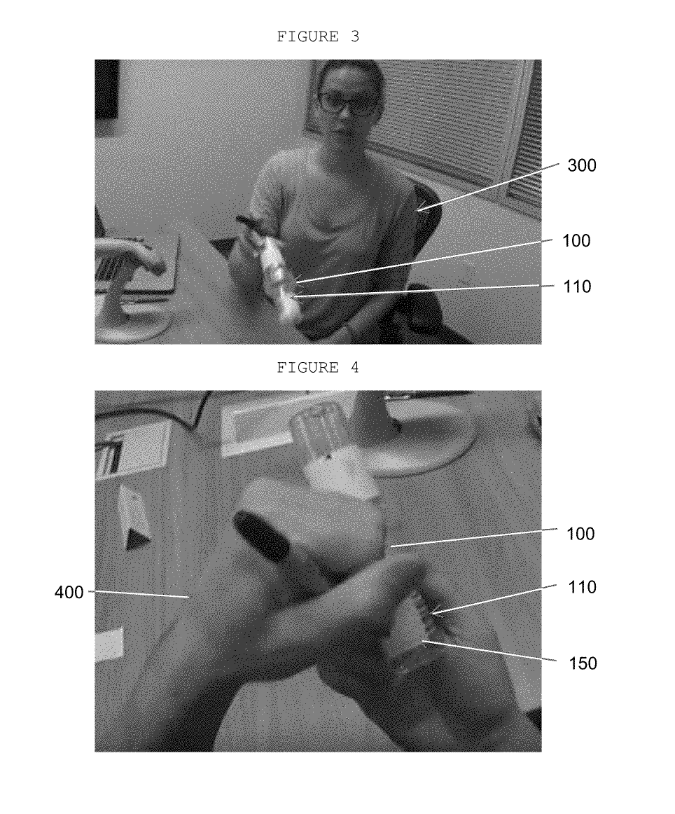

FIG. 1 illustrates an embodiment of the label 110, which is attached to a surface of sample container 100 and includes two sides, where a first portion 130 is colored one color, for example blue, and a second portion 120 is colored another color, for example, white; each portion includes a space 150 for the patient to place their signature, as well as barcode 140 containing the patient's information. The label 110 may contain branding on its surface. The first portion 130 is detachable from the rest of the label; FIG. 2 illustrates label 110 after first portion 130 has been detached. The area of the label 200 exposed after the detachment of side 130 may contain a message, such as a warning or instructions, or illustration. This label consolidates the functionality of the current method's patient information strip and the patient's signatures on the cup and tamper strip.

Label and Tamper-Proof Seal

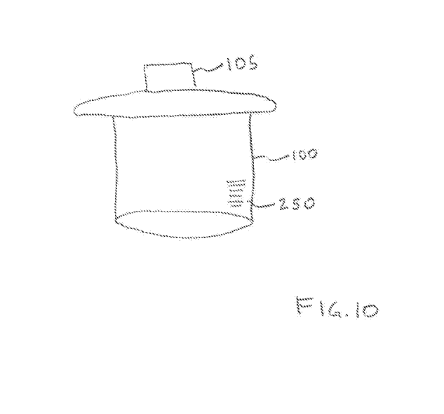

The label 110 may also function as a tamper-proof device, thereby eliminating the separate step of tamper-proofing the vessel and, in some cases, signing the tamper strip or label. As illustrated in FIGS. 8-9, the label 210 may have an end 220 that is releasable from the vessel 100, that has an accordion or fold-out structure that can be extended to wrap around the top 105 of the vessel 100, resealing in a way that re-opening the vessel 100 would break the structure 220 to indicate if the vessel 100 has been reopened.

The label 210 may include a hanging end 230 that is not attached to the vessel 100, which end can be extended and/or reattached to the vessel 100 to act as a tamper strip for the vessel 100. The label 210 may include a hanging end 230 that is not attached to the vessel 100, which end 230 can be extended and includes a peel away film or paper 240 that exposes an area of the end of the label that can reattach to the vessel 100 to act as a tamper strip for the vessel 100.

This releasable label 210 may act as a tamper strip and does not require extra identifying information.

In one embodiment, the label 210 may include a bar code or other mechanism 250 to record information related to the sample.

Built-in Label

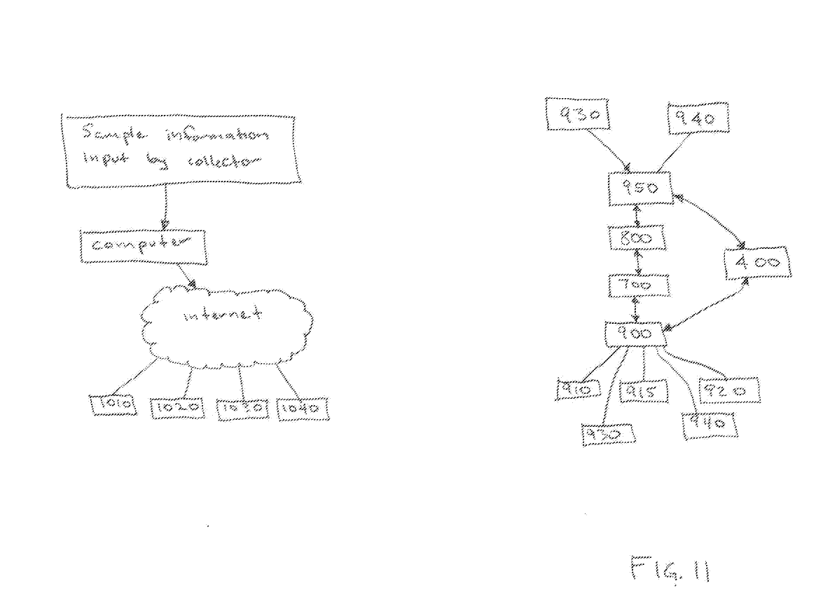

Along these lines, as seen in FIG. 10, the sampling device may include a label, or the ability to record the information that would otherwise be captured on a traditional label, as an integral part of the sample vessel 100. The sample vessel 100 may include an area to record the identifying information related to the sample, which area is built-onto or embedded onto the vessel 100. This takes the form of a pre-attached bar code or a chip 250, which code or chip can later be assigned the appropriate identifying information. This mechanism 260 to store and identify information may be 3-D printed onto the vessel or embedded into the vessel, such as by forming the vessel with the information therein. This mechanism to store and identify information may pre-attached to the vessel by an adhesive or otherwise pre-printed on the vessel.

The sample vessel may be a sponge that is attached to a stick. A person of ordinary skill in the art would understand that the labels described herein would be sized to be appropriate for the collection vessel at issue and/or for the information that need be captured on the label. Related hereto, U.S. Patent Pub. No. 2017/0143314 is incorporated by references as if fully set forth herein.

Sample Labeling Process

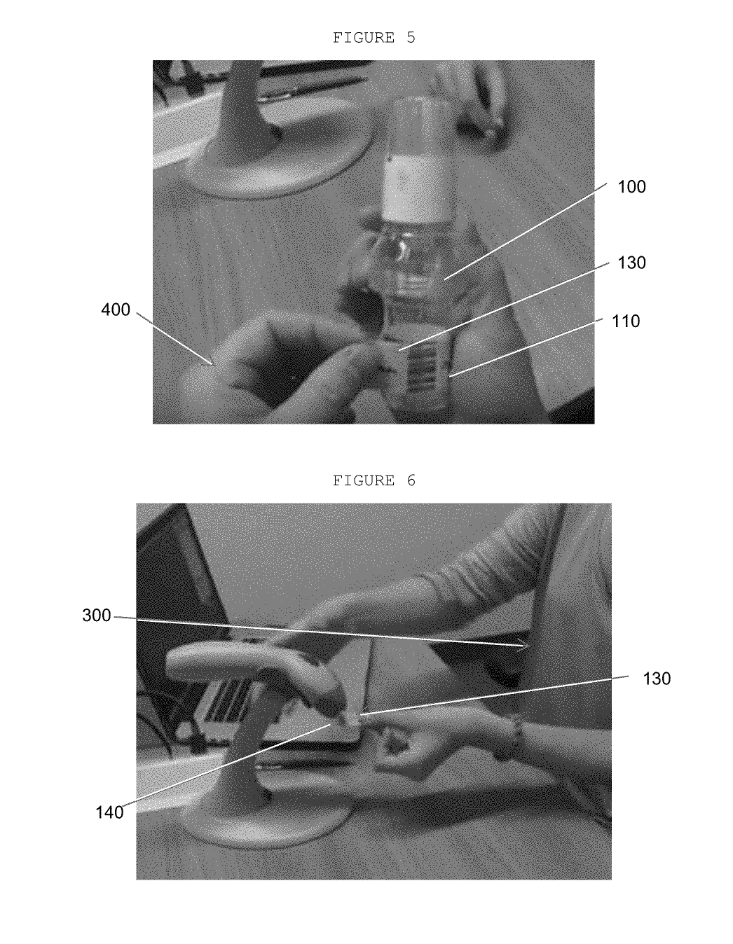

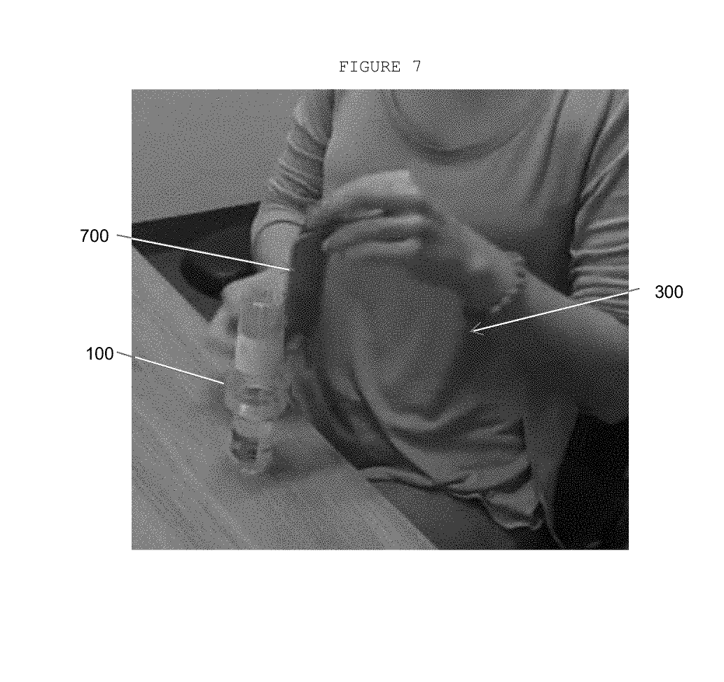

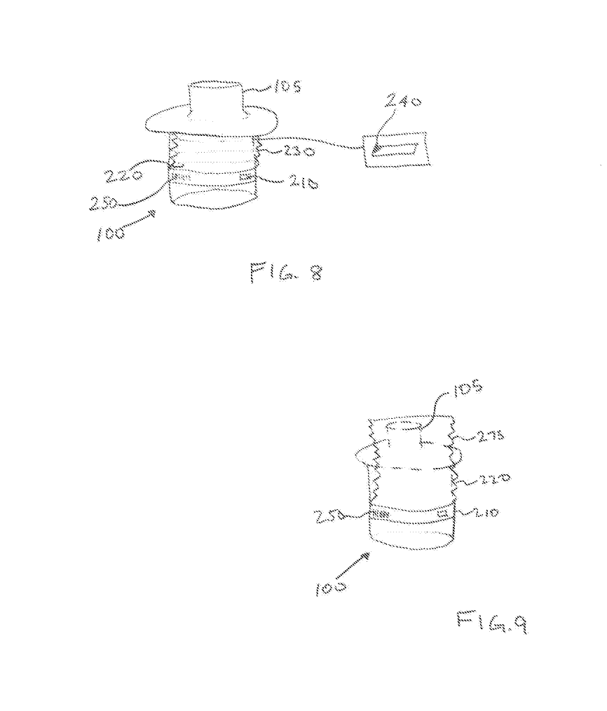

By making use of the double-barcode label, it is possible to perform a more streamlined process of labeling and sealing the sample cup by following a process: First, as illustrated in FIGS. 3-7, the collector 300 hands the patient sample cup 100 upon which is attached label 110; The collector 300 then instructs the patient 400 to, as shown in FIG. 8, initial the two boxes 150 on each portion of label 110 attached to the sample cup 100, and, as shown in FIG. 5, detach the first portion 130 of the label 110. Next, as shown in FIG. 6, the collector 300 scans the patient's information into the digital or other records system using the barcode 140 on the detached first portion 130 of the label 110. FIG. 7 shows the last step of the process, wherein the collector 300 seals the cup 100 with a tamper strip 700 (or other tamper strip embodiments described herein), which adheres to the cap and sides of the sample cup and tears if the cup is opened, thereby alerting medical personnel to the possibility of tampering.

It should be noted that the patient does not need to fill the sample cup during this process. Through this simplified process, the patient need only place two signatures on one labeling element, rather than three signatures on two separate labeling elements as required by the current method. Furthermore, this eliminates the current method's need for an additional adhesive strip containing the patient's information and a scannable barcode, Further, there is no need to visually confirm that the information on each element matches the information on the others, as both labeling elements in the proposed method come from the same label, and already contain the same information. Lastly, there is no need for the collector to transfer the sample from a cup to a labeled specimen holder, another step which introduces potential error into the sample taking process. As a result, the proposed process has fewer steps where mistakes could be made, and thereby decreases the likelihood of user error and increases the authenticity of the labels.

As shown in FIG. 9, as a further improvement, if the patient is using the label and tamper-proof seal 210, the patient or sample collector does not need to potentially label yet another identifying strip, eliminating another source of human error. Further, having the label and the tamper-seal be one in the same further simplifies the sample process, eliminating sources of error and/or increases the integrity of the sample. In this case, the collector 300 hands the patient sample cup 100 upon which a label 210 has been attached. The patient signs the label 210, and the collector 300 scans the patient's information into the digital or other records system using the barcode or identifying mechanism 250 on the label 210. The collector then extends the label 210, using the hanging end 230, peels away an adhesive 240 on the hanging end 230, and then reattaches the label 210 across the top of the container as a tamper strip.

As shown in FIG. 10, if the patient is using the built-in label, yet another step of the process, that of matching and attaching labels to sample vessels, is eliminated, thereby further streamlining the sample process, and further reducing error and increasing confidence in the results.

As shown in FIG. 11, the patient may track the chain of custody of the sample, as well as the status of the testing process, through a computer, web-based program 800 and a coordinating mobile device application (“app”) 900, such as a web-enabled application, that provides information to a user, accessible on his mobile device or computer. This program 800 and app 900 could be accessed on any kind of a computer hardware system, the computer having a processor and memory and a means for a user to input information, such as a keyboard or a touch screen, and a means for the computer to give output, such as a display screen. This program and app may use the identifying information on the sample vessel, for example the bar code or chip, and may be configured to receive data from, and supply data to, a user, including information that would inform the user of the location and status of the sample, thereby adding another way to follow and secure the chain of custody of the sample. The user may customize the data stored, sent and received by the program and/or app. This existence of such a program and/or app may improve a patient's comfort and confidence with the process, and help avoid any problems resulting from a less transparent chain of custody process.

Additionally, the program 800 and/or app 900 may execute and/or operate with one or more security processes that are configured to control access to the functions of the app. For example, the program and/or app may execute a security process that requires a user to provide a valid username and password (or other form of identification) to access the features of the app. Further, the app could include certain alerts based on the status of the sample.

The program and/or app may include one or more user interfaces that allow a user to exchange information with the program and/or app, such as through display. User interface allows a user to request data in the form of commands or queries that are processed by business logic/transaction engine. The business logic/transaction engine 850 may be software, when executed by the user through the computer, manages one or more tasks (i.e., processes) consistent with certain features related to the present invention. In one aspect, these tasks may include a location tracker, a sample status identifier, other features task, and alerts task. While the invention has been described with reference to the embodiments above, a person of ordinary skill in the art would understand that various changes or modifications may be made thereto without departing from the scope of the claims.

We claim the following:

1. A method of labeling and sealing a container for medical samples comprising the steps of:
a) supplying a patient a sample cup including an attached label comprising a detachable portion and a tamper strip portion, wherein the detachable portion is detachable from the attached label, and wherein both the detachable portion and the temper strip portion comprise a barcode for storing patient information;
b) instructing the patient to initial the detachable portion and the tamper strip portion, and to remove the detachable portion;
c) scanning the patient information into a digital records system; and
d) sealing the sample cup with the tamper strip portion.

2. A method of labeling and sealing a container for medical samples comprising the steps of:
a) supplying a patient a sample cup comprising a pre-attached a mechanism for storing patient information;
b) scanning the patient's information into a digital records system using the mechanism on the label; and
c) sealing the sample cup with a tamper strip.

3. The method of claim 2, further comprising tracking the status of the medical sample by means of a computer system program that is coupled to a wireless data collection system.

4. The method of claim 3, wherein the computer system program further includes and communicates with a computer application that is accessible on a user's personal mobile device.

5. The method of claim 4, wherein the computer application informs the patient of the status of the sample testing procedure.

6. A label for use to identify a patient sample comprising:
an inner surface, an outer surface, and a releasable end, wherein the outer surface includes a mechanism to store information to identify a patient sample, wherein the inner surface is partially attached to an outer surface of a patient sample container, and wherein the releasable end is configured to be extended away from the outer surface of the container and reattached to a top of the container as a tamper strip.

7. The label of claim 6, wherein the releasable end is configured like an accordion prior to extension.

8. The label of claim 6, wherein the releasable end is not fully attached to the outside of the container.

9. The label of claim 6, wherein the mechanism is a bar code.

10. The label of claim 6, wherein the mechanism is integrally attached to the container.

11. The label of claim 10, wherein the mechanism is 3D-printed onto the container.
